(19) United States
(12) Patent Application Publication
(10) Pub. No.: US 2024/0065915 A1
(43) Pub. Date: Feb. 29, 2024

(54) SEALED POSITIONERS THAT MAINTAIN INTERNAL AIR PRESSURE IN DIFFERENT ENVIRONMENTS

(71) Applicant: Greenwood Marketing, LLC, Bedford, NY (US)

(72) Inventors: William J. Purdy, Bedford, NY (US); Robert Purdy, Bedford, NY (US)

(21) Appl. No.: 18/239,750

(22) Filed: Aug. 29, 2023

Related U.S. Application Data
(60) Provisional application No. 63/402,033, filed on Aug. 29, 2022.

(51) Int. Cl. A61G 13/12; A61M 21/00

(52) U.S. Cl. CPC A61G 13/1265; A61M 21/00; A61M 2021/0022; A61M 2240/00

(57) ABSTRACT

A sealed positioner for positioning subjects or their body parts includes a filter that maintains an internal air pressure and, thus, a particular “feel,” of the positioner as it is moved from one environment to another. The sealed positioner can maintain its internal air pressure and “feel” regardless of the environment (e.g., elevation, atmospheric pressure, etc.) in which it is used. Methods in which the internal air pressure and feel of a sealed positioner are maintained as the sealed positioner is moved from one environment to another are also disclosed.

### CROSS-REFERENCE TO RELATED APPLICATION

A claim for priority us hereby made to the Aug. 29, 2022 filing date of U.S. Provisional Patent Application 63/402,033, titled SEALED POSITIONERS THAT MAINTAIN INTERNAL AIR PRESSURE IN DIFFERENT ENVIRONMENTS (“the '033 Provisional Application”). The entire disclosure of the '033 Provisional Application is hereby incorporated herein.

### TECHNICAL FIELD

This disclosure relates generally to sealed positioners for positioning subjects or their body parts and, more specifically, to sealed positioners with configurations that enable the sealed positioners to maintain an internal air pressure and, thus, a particular “feel,” as they are moved from one environment to another. More specifically, this disclosure relates to sealed positioners with configurations that enable the sealed positioners to maintain their internal air pressure and “feel” regardless of the environment (e.g., elevation, atmospheric pressure, etc.) in which they are used. This disclosure also relates to methods in which the internal air pressure and feel of a sealed positioner are maintained as the sealed positioner is moved from one environment to another.

### RELATED ART

Positioners are often used in medical settings to maintain a position of a subject or a subject's body part. For example, medical positioners have been used in neonatal intensive care units (NICUs) to position newborn infants, including premature infants. Medical positioners have also been used to help stabilize body parts that are undergoing medical treatment or recuperating from surgery.

Since medical positioners are typically used against a subject or their body part, the sensation given by the medical positioner, or its “feel,” against the subject's body is usually important. In the example of the NICU, experience has shown that medical positioners that provide the feel of the prenatal environment (i.e., within the mother's womb) enable premature infants to experience normal development, which may not occur with medical positioners that do not provide the feel of the prenatal environment.

In embodiments where the shapeable elements of a medical positioner are sealed, the ability of the medical positioner to provide a subject with a particular sensation, or “feel,” may depend on the atmospheric pressure where the medical positioner is used. This is particularly true for medical positioners that contain air. Such a medical positioner may have an internal air pressure that enables a sealed cover of the medical positioner to be relatively loose at lower altitudes (e.g., sea level, etc.), but whose internal air pressure may increase, undesirably, causing the sealed cover to expand when the medical positioner is subjected to lower pressure environments (e.g., at higher altitudes, during air travel, etc.).

### SUMMARY

A positioner of this disclosure, which may comprise a cushion, is sealed. The sealed positioner may include a deformable material, a container for the deformable material, and a sealed cover with at least one filter. Together, the deformable material and the container may define a shapeable core of the positioner. Optionally, the positioner may include an intermediate material between the container and the sealed cover. Together, the sealed cover, the filter, and the shapeable core, along with relative configurations of the sealed cover and the shapeable core and any optional intermediate material, may provide the sealed positioner with a particular feel.

The deformable material may comprise a moldable composition, a loose fill material that may hold a shape when placed under a load, or the like. While particles or parts of the deformable material may themselves be resilient, the deformable material has a whole may substantially lack resiliency or lack resiliency.

The container may hold the deformable material. In embodiments where the deformable material comprises a loose fill material, the container may comprise a bag (e.g., a breathable bag, etc.). In embodiments where the deformable material comprises a moldable composition (e.g., lubricated microspheres, microspheres in a fluid, etc.), the container may comprise a sealed bladder.

The optional intermediate material is located outside of the container. The intermediate material may comprise a cushioning material, a quantity of loose fill material that may hold a shape when placed under a load (i.e., a compressible fill material through which air may flow) (e.g., compressible beads, polyfiber, foam rubber, a spacer fabric, etc.), or the like. The intermediate material may be located between the shapeable core and the interior surface of the sealed cover. In some embodiments, the intermediate material may fill only a portion of the interior of the sealed cover; for example, it may be located on only one side (e.g., a base, etc.) of the shapeable core or on a plurality of sides, but not all of the sides, of the shapeable core. Alternatively, the intermediate material may be located completely around the shapeable core.

As another option, the intermediate material may comprise gas or a mixture of gases (e.g., air, etc.) within the interior of the sealed cover, between at least part of the shapeable core and the interior surface of the sealed cover.

The sealed cover may include an exterior surface and an interior surface. The interior surface of the sealed cover may define an interior of the sealed cover. The interior of the sealed cover may have at least one property (e.g., a volume, an air pressure, etc.), which may comprise a predetermined property of the interior. The interior of the sealed cover includes holds the container and the optional cushioning material or fill material. The sealed cover may be impermeable to fluids and air, and it may provide a barrier that prevents microorganisms from passing therethrough. The sealed cover may comprise a hermetically sealed cover.

The filter(s) in the sealed cover may maintain a seal of the sealed cover and enable air to flow into and out of the interior of the sealed cover without affecting the at least one property of the interior of the sealed cover and without compromising the integrity of the sealed cover. The at least one property that is maintained within the interior of the sealed cover as air flows into and/or out of the filter may comprise a volume of the interior of the sealed cover, an air pressure within the interior of the sealed cover, a maximum particle size within the interior of the sealed cover, and/or the like. While the filter(s) may allow air to pass into and out of the interior of the sealed cover, the filter(s) may be impermeable to liquids and prevent(s) microorganisms from passing into or out of the interior of the sealed cover.

The filter enables the sealed positioner to be used in a variety of different environments, at a plurality of different elevations and/or pressures (e.g., atmospheric pressures, etc.) while maintaining at least one property of the sealed positioner, such as a feel of the sealed positioner. For example, the filter may enable the sealed positioner to retain or substantially retain (e.g., to within about 10%) its intended volume at a plurality of different elevations. As another example, the filter may enable the sealed positioner to maintain or substantially maintain (e.g., to with about 10%) its intended pressure at a plurality of different elevations.

Other aspects of this disclosure, as well as features and advantages of various aspects of the disclosure, should be apparent to those of ordinary skill in the art through consideration of the ensuing description, the accompanying drawings, and the appended claims.

### BRIEF DESCRIPTION OF THE DRAWINGS

In the drawings:

FIG. 1 is a perspective view of an embodiment of a sealed positioner that includes a shapeable core and a cover with a filter;

FIG. 2 is a cutaway perspective view of the embodiment of the sealed positioner shown in FIG. 1, showing internal elements of the sealed positioner;

FIG. 3 is a cross-sectional representation of the embodiment of the sealed positioner shown in FIG. 1;

FIG. 4 is a cutaway perspective view of another embodiment of a sealed positioner that includes a shapeable core, an intermediate material, and a cover with a filter, with the intermediate material being positioned between only a portion (e.g., a base, etc.) of the shapeable core and the cover;

FIG. 5 is a cross-sectional representation of the embodiment of the sealed positioner shown in FIG. 4;

FIG. 6 is a cutaway perspective view of yet another embodiment of a sealed positioner that includes a shapeable core, an intermediate material, and a cover with a filter, with the intermediate material surrounding the shapeable core;

FIG. 7 is a cross-sectional representation of the embodiment of the sealed positioner shown in FIG. 6;

FIG. 8 is a cutaway perspective view of still another embodiment of a sealed positioner that includes a shapeable core and a cover with a filter, with a container of the shapeable core also including a filter; and

FIG. 9 is a cross-sectional representation of the embodiment of the sealed positioner shown in FIG. 8.

### DETAILED DESCRIPTION

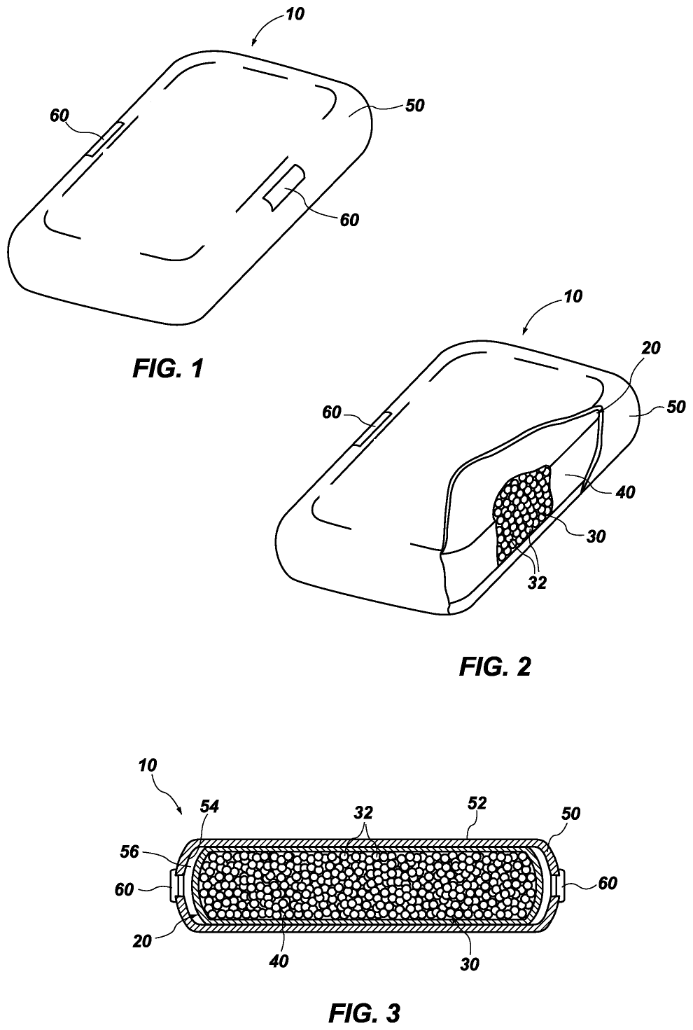

With reference to FIGS. 1-3, an embodiment of a sealed positioner 10 is depicted. The sealed positioner 10 includes a shapeable core 20 and a sealed cover 50 with a filter 60. The shapeable core 20 includes a deformable material 30 and container 40 for the deformable material 30.

The shapeable core 20 may be deformable. In some embodiments, the shapeable core 20 may also be compressible. In some embodiments, the deformable material 30 of the shapeable core 20 may comprise a material that flows, or a “flowable material.” The container 40 of the shapeable core 20 may comprise a pliable container (e.g., bag, bladder, etc.).

The deformable material 30 may comprise a dry, flowable material. The dry, flowable material may include particles 32 that can flow freely relative to one another. A volume of the dry, flowable material, or a collection of the particles 32, may hold a shape when placed under a load. As an example, the particles 32 of the dry, flowable material may comprise compressible beads. The compressible beads may be compressed from round (e.g., spherical, spheroid, etc.) shapes to more flattened shapes. The compressible beads may be porous. In some embodiments, the compressible beads may comprise foam beads. More specifically, the foam beads may comprise expanded polytheylene beads. In a specific example, the foam beads may comprise the polyethylene beads available from JSP International Group, Ltd, of Wayne, Pennsylvania, such as the white polyethylene beads having a density of 14.4 g/L to 18.0 g/L and sold as ARPAK® 4313 polyethylene beads. Other materials that can be foamed or expanded and may then be compressed may also be used as the compressible beads.

The compressible beads may have an uncompressed state and a compressed state. When uncompressed, the compressible beads may flow freely throughout the container 40. When compressed, the compressible beads may flatten as they are forced against each other. The coefficient of friction of the surfaces of the compressed compressible beads may prevent them from flowing relative to one another, enabling the compressible beads to define a semi-rigid layer. Thus, the compressible beads may define a conformable layer that may transition between flowable and semi-rigid states.

As an alternative to compressible beads, the deformable material 30 may comprise other compressible media that can function in the manner describe above can be used. For example, fibers (e.g., polyfiber, microfiber, etc.) that flow when uncompressed but provide a more rigid structure when compressed may be used. In some embodiments, a material that enables temperature regulation (e.g., a phase change material, etc.) may be used in place of the compressible medium or in addition to compressible medium.

In embodiments where the deformable material 30 comprises a dry, flowable material, air may flow through the container 40 of the shapeable core 20. As an example, the container 40 may comprise a fabric bag or the like.

As another example, the deformable material 30 of the shapeable core 20 may comprise a fluidized medium, such as microspheres (e.g., hollow microspheres, etc.) dispersed throughout a lubricant (e.g., liquid silicone, etc.). The container 40 for such a fluidized medium may comprise a sealed polyurethane bladder or the like. While a shapeable core 20 with a deformable material 30 that comprises a fluidized medium may be compressible due to the ability of the fluidized medium to flow from one location to another within the container 40. The fluidized medium of such a shapeable core 20 may be substantially incompressible.

The sealed cover 50 may be defined from a material that is pliable. The pliability of the material may enable it to conform to the shape of a body part of the subject, to the shape of a subject, and/or to a surface on which the sealed positioner 10 is to rest while used to support the body part or the subject. The material of the sealed cover 50 may be impermeable (e.g., to fluids, including air and liquids; etc.). The sealed cover 50 may be defined and/or sealed by any of a variety of suitable processes, which may, of course, depend on the material from which the sealed cover 50 is defined. Some non-limiting examples of processes for defining and/or sealing the sealed cover 50 include heat-sealing, radiofrequency (RF) welding, ultrasonic sewing, and conventional sewing to secure edges of the material together to define and/or seal the sealed cover 50.

The sealed cover 50 includes an outer surface 52 and an interior surface 54. The interior surface 54 defines an interior 56 of the sealed cover 50.

The outer surface 52 of the sealed cover 50 may have a texture that imparts it with certain properties. For example, a portion of the outer surface 52 may prevent it from slipping when placed on another surface (e.g., it may comprise a non-slip material, etc.). As another example, at least a portion of the outer surface 52 of the sealed cover 50 may have texture that is comfortable when placed against a portion of a subject's body. As yet another example, at least a portion of the outer surface 52 of the sealed cover 50 may provide certain benefits (e.g., enables air to flow between it and a subject, increases oxygen levels in the subject's skin, stimulates blood flow in the subject's skin, etc.).

The filter 60 may have a configuration that enables it to maintain the at least one property within the interior 56 of the sealed cover 50, such as a predefined or predetermined volume, pressure, and/or maximum particle size. Without limitation, the filter 60 may enable air to flow through it while limiting the size of particles that may flow through it. In some embodiments, the filter 60 may be an antimicrobial filter, which may exclude bacteria and even viruses from the interior of the sealed cover 50. An example of such a filter 60 is the PNEUMAPURE® filter available from Pneuma Pure L.P. Limited and disclosed by U.S. Pat. No. 8,561,233 (“the '233 Patent”) and 8,950,028 (“the '028 Patent”), the entire disclosures of which are hereby incorporated herein. Such a filter 60 may be assembled with and secured to the sealed cover 50 in the manner and orientation described by the '233 Patent and the '028 Patent. Such a filter 60 may maintain a predetermined volume, air pressure, and/or maximum particle size within the interior 56 of the sealed cover 50 while preventing microorganisms (e.g., bacteria, fungi, mold, and even some viruses, etc.) from entering into the interior 56 of the sealed cover 50.

In some embodiments, the sealed cover 50 may be positioned directly and in substantial contact with an outer surface 22 of the shapeable core 20. In other embodiments, a gas or mixture of gases (e.g., air, etc.) may be located between the shapeable core 20 and the interior surface 54 of the shapeable cover 50. The gas(es) may occupy a predefined or predetermined volume of space between the shapeable core 20 and the interior surface of the shapeable cover 50. The gas(es) may impart the interior 56 of the sealed cover 50 with a predefined or predetermined pressure (e.g., air pressure, etc.).

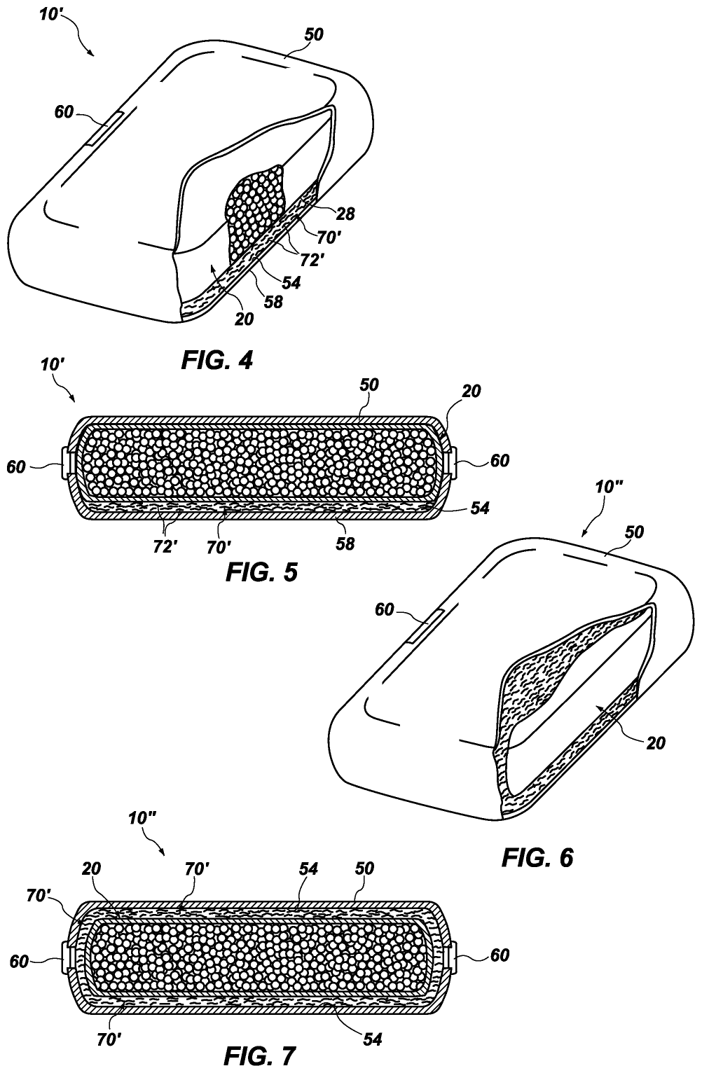

Turning now to FIGS. 4 and 5, another embodiment of a sealed positioner 10′ is illustrated. The sealed positioner 10′ includes a shapeable core 20 and a sealed cover 50 with a filter 60, as described previously herein with respect to FIGS. 1-3. In addition, the sealed positioner 10′ includes an intermediate material 70′ between a portion of the shapeable core 20 and the interior surface 54 of the sealed cover 50. More specifically, the intermediate material 70′ may be located between a base 28 of the shapeable core 20 and a base 58 of the sealed cover 50.

The intermediate material 70′ may comprise a dry, flowable material. The dry, flowable material may include particles 72′ that can flow freely relative to one another. A volume of the dry, flowable material, or a collection of the particles 72′, may hold a shape when placed under a load. As an example, the particles 72′ of the dry, flowable material may comprise compressible beads, as described previously herein. Alternative, nonlimiting examples of intermediate materials 70′ include polyfiber, foam rubber, spacer fabrics, and the like.

FIGS. 6 and 7 show another embodiment of a sealed positioner 10″. The sealed positioner 10″ includes a shapeable core 20 and a sealed cover 50 with a filter 60, as described previously herein with respect to FIGS. 1-3, as well as an intermediate material 70′, as described previously herein with respect to FIGS. 4 and 5. The intermediate material 70′ may surround or substantially surround the shapeable core 20. More specifically, the intermediate material 70′ may be located or locatable between all of the surfaces of the shapeable core 20 and the interior surfaces 54 of the sealed cover 50.

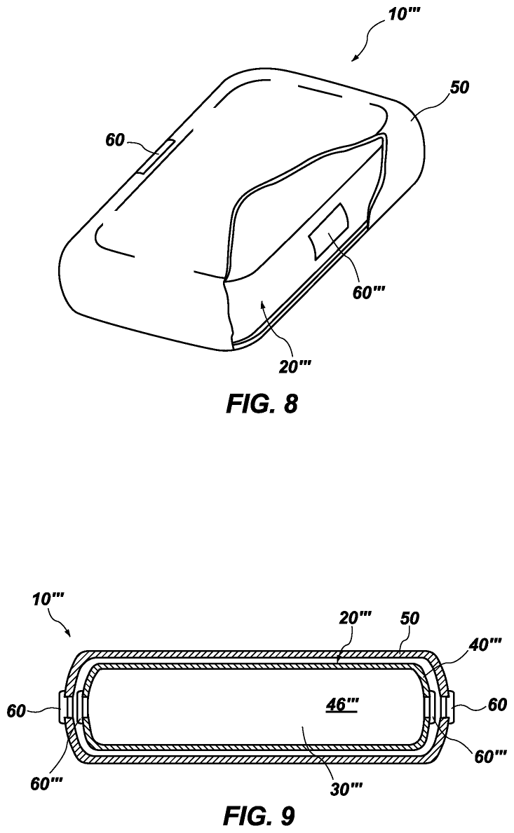

Turning now to FIGS. 8 and 9, another embodiment of a sealed positioner 10′″ is depicted. The sealed positioner 10′″ includes a shapeable core 20′″ and a sealed cover 50 with a filter 60. The shapeable core 20′″ may be configured substantially as described previously herein with reference to FIGS. 1-3, but with the addition of a filter 60′″ to the container 40′″ of the shapeable core 20′″. The filter 60′″ may have a configuration similar to that described previously herein with reference to FIGS. 1-3

In embodiments where the deformable material 30 of the shapeable core 20′″ includes a fluid (e.g., liquid, gel, etc.), such as microsphere dispersed throughout a liquid medium, the filter 60′″ may include an oleophobic coating on its filter membrane (e.g., in place of a hydrophobic coating, etc.) and be oriented in a direction opposite from that described by the '233 Patent and the '028 Patent, with its with its filter membrane or its optional backing facing an interior 46′″ of the container 40′″ and the deformable material 30′″ therein. Such an arrangement may enable the filter 60′″ to moderate the volume, pressure, etc., of any ambient air in the deformable material 30′″.

The sealed positioner 10′″ may lack an intermediate material 70′ (FIGS. 4 and 5) or any gas(es) between the shapeable core 20′″ and the sealed cover 50, or it may include gas(es), as shown in and described with reference to FIGS. 1-3 or an intermediate material 70′, as shown in and described with reference to FIGS. 4 and 5 or FIGS. 6 and 7.

With returned reference to FIGS. 1-3, and more specifically, in embodiments where the deformable material 30 of the shapeable core 20 comprises compressible beads and the container 40 of the shapeable core 20 is breathable, the compressible beads may be compressed under the weight of an object, such as a body part of a subject (e.g., a head, a back, a torso, a hip or hips, a leg or legs, a foot or feet, etc.) or a subject as the body part or the subject rests on the shapeable core 20. The container 40 and the deformable material 30 therein may contour to the shape of the object and/or to a surface that supports the shapeable core 20. In embodiments wherein the deformable material 30 comprises beads and the container 40 is breathable, when compressed, air may be expelled from interstitial spaces between the compressible beads and from the container 40. As friction increases between the compressed beads, the compressible beads may define a semi-rigid layer that conforms to a shape of the object and/or to a shape of the surface that supports the compressible layer and/or the element of which the compressible layer is a part. A volume of compressible beads within the container 40 may be sufficient to enable compressible beads to remain between the object and a surface upon which the shapeable core 20 rests.

Upon removal of a compressive force (e.g., by lifting the body part, the subject, etc.), the compressible beads may expand when compressive force is removed, pulling air into the container 40 and the interstices between the compressible beads. As the compressible beads return to their initial shape, the surface contact and, thus, the friction between adjacent compressible beads are reduced. Thus, the beads may again flow relative to one another.

A method for transporting a sealed positioner 10 (or any other embodiment of sealed positioner) includes providing a sealed positioner 10, moving the sealed positioner 10 from a first environment to a second environment, and maintaining a predetermined pressure within the sealed positioner 10. In addition, such a method may include maintaining a seal of a sealed cover 50 of the sealed positioner 10 and at least one property of an interior 56 of the sealed cover 50 while moving the sealed positioner 10 from the first environment to the second environment.

Providing the sealed positioner 10 may include providing a sealed positioner 10 that includes a shapeable core 20 within an interior of a sealed cover 50. The interior 56 of the sealed cover 50 (e.g., a portion of the interior between the shapeable core 20 and the inner surface 54 of the sealed cover 50, an entire interior 56, etc.) may have a predetermined pressure and/or a predetermined volume.

Moving the sealed positioner 10 from the first environment to the second environment may include moving the sealed positioner 10 from a first environment having a first pressure (e.g., a first atmospheric pressure at a first elevation, etc.) to a second environment having a second pressure (e.g., a second atmospheric pressure at a second elevation, within an aircraft (e.g., a medical transport helicopter, an airplane, etc.), etc.). The first pressure and the second pressure may be different from each other.

Maintaining the predetermined pressure may include maintaining the predetermined pressure within the interior 56 of the sealed cover 50 regardless of the environment, elevation, and/or pressure within which the sealed positioner is located. For example, the predetermined pressure may be the same while the sealed positioner 10 is located within the first environment, while moving the sealed positioner from the first environment to the second environment, and while the sealed positioner is located within the second environment. The predetermined pressure may be maintained by enabling gas to flow through the filter(s) 60 in the sealed cover 50. Gas may flow from the interior 56 of the sealed cover to a location outside the sealed cover 50 while moving the sealed positioner 10 from the first environment to the second environment, with the first pressure exceeding the second pressure. Alternatively, gas may flow from a location outside the sealed cover 50 into the interior of the sealed cover 50 while moving the sealed positioner 10 from the first environment to the second environment, with the first pressure being less than the second pressure. In any event, by maintaining the predetermined pressure within the interior 56 of the sealed positioner 10, the sealed positioner 10 may provide a subject with the same sensation, or feel, regardless of the environment in which the sealed positioner 10 is used.

Although the preceding disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A sealed positioner, comprising:
a sealed cover defining an exterior of the sealed positioner and including an interior with a substantially constant air pressure;
a filter in the sealed cover that enables air to flow into and out of the interior of the sealed cover while maintaining a seal of the sealed cover and the substantially constant air pressure within the interior of the sealed cover; and
a shapeable core within the interior of the sealed cover.

2. The sealed positioner of claim 1, wherein the filter comprises an antimicrobial filter.

3. The sealed positioner of claim 1, wherein the sealed cover is defined from an impermeable material.

4. The sealed positioner of claim 1, wherein the shapeable core comprises a moldable material.

5. The sealed positioner of claim 4, wherein the moldable material comprises compressible beads.

6. The sealed positioner of claim 5, wherein the compressible beads comprise expanded polyethylene beads.

7. The sealed positioner of claim 4, wherein the moldable material comprises particles dispersed throughout a fluid medium.

8. The sealed positioner of claim 7, wherein the particles comprise hollow microspheres and the fluid medium comprises a liquid silicone.

9. A sealed positioner, comprising:
a shapeable core;
a sealed cover including an interior surface defining an interior of the sealed cover, the interior carrying the shapeable core and having a predetermined pressure and maximum particle size between the shapeable core and the interior surface of the sealed cover;
a filter in the sealed cover that enables air to flow into and out of the interior of the sealed cover while maintaining a seal of the sealed cover, the predetermined pressure, and the maximum particle size.

10. The sealed positioner of claim 9, wherein the filter excludes bacteria from the interior of the sealed cover.

11. The sealed positioner of claim 10, wherein the filter further excludes viruses from the interior of the sealed cover.

12. The sealed positioner of claim 9, wherein the sealed cover is defined from an impermeable material.

13. The sealed positioner of claim 9, wherein the shapeable core comprises a moldable material.

14. The sealed positioner of claim 13, wherein the moldable material comprises compressible beads.

15. The sealed positioner of claim 13, wherein the moldable material comprises particles dispersed throughout a fluid medium.

16. A method for transporting a sealed positioner, comprising:
providing a sealed positioner that includes a shapeable core within an interior of a sealed cover, with a predetermined pressure between the shapeable core and an inner surface of the sealed cover;
moving the sealed positioner from a first environment to a second environment, the first environment having a first pressure, the second environment having a second pressure, and the first pressure and the second pressure being different from each other;
maintaining the predetermined pressure between the shapeable core and the inner surface of the sealed cover while the sealed positioner is located within the first environment, while moving the sealed positioner from the first environment to the second environment, and while the sealed positioner is located within the second environment by enabling gas to flow through a filter in the sealed cover; and
maintaining a seal of the sealed cover and at least one property of the interior of the sealed cover while moving the sealed positioner from the first environment to the second environment.

17. The method of claim 16, wherein enabling gas to flow through the filter comprises enabling gas to flow from the interior of the sealed cover to a location outside the sealed cover while moving the sealed positioner from the first environment to the second environment, with the first pressure exceeding the second pressure.

18. The method of claim 16, wherein enabling gas to flow through the filter comprises enabling gas to flow from a location outside the sealed cover into the interior of the sealed cover while moving the sealed positioner from the first environment to the second environment, with the first pressure being less than the second pressure.

19. The method of claim 16, wherein moving the sealed positioner from the first environment to the second environment comprises moving the sealed positioner from a first elevation to a second elevation.

20. The method of claim 16, wherein maintaining the predetermined pressure comprises maintaining a feel of the sealed positioner within the first environment and the second environment.
